(19) United States
(12) Patent Application Publication
(10) Pub. No.: US 2020/0330429 A1
(43) Pub. Date: Oct. 22, 2020

(54) METHOD OF TREATING TRUNCAL ACNE WITH TRIFAROTENE

(71) Applicant: Nestlé Skin Health SA, Lausanne (CH)

(72) Inventors: Alessandra ALIO-SAENZ, Northlake, TX (US); Michael GRAEBER, Flower Mound, TX (US); Faiz AHMAD, Flower Mound, TX (US)

(21) Appl. No.: 16/854,716

(22) Filed: Apr. 21, 2020

Related U.S. Application Data
(60) Provisional application No. 62/837,054, filed on Apr. 22, 2019.

(51) Int. Cl. A61K 31/40; A61P 17/10; A61K 9/00; A61K 9/06

(52) U.S. Cl. CPC A61K 31/40; A61K 9/06; A61K 9/0014; A61P 17/10

(57) ABSTRACT

Disclosed herein are methods for selectively treating acne vulgaris of the face and/or trunk in a subject, pharmaceutical compositions for use in the treatment of acne vulgaris of the face and/or trunk in a subject, uses of trifarotene or an equivalent thereof in the manufacture of a medicament for the treatment of acne vulgaris of the face and/or trunk in a subject.

### CROSS-REFERENCE TO RELATED APPLICATION

This application claims priority under 35 U.S.C. § 119(e) to U.S. Provisional Application No. 62/837,054 filed Apr. 22, 2019, the entire contents of which are incorporated herein by reference.

### FIELD

Described herein are methods for treating a subject having facial and truncal acne, pharmaceutical compositions for use in the treatment of facial and truncal acne, uses of trifarotene or an equivalent thereof in the manufacture of a medicament for the treatment of facial and truncal acne.

### BACKGROUND

The following discussion is provided to aid the reader in understanding the disclosure and is not admitted to describe or constitute prior art thereto.

Acne vulgaris (AV) is one of the most common skin diseases, and it has a multifactorial pathogenesis that centers on pilosebaceous units. Because the pathophysiology and clinical presentations of facial and truncal acne are considered to be similar, clinicians often apply the same therapeutic approach for facial and non-facial lesions despite lack of evidence in truncal AV. A variety of treatment options are currently available for AV, but they have not been rigorously studied in truncal skin diseases, such as truncal acne. Truncal acne refers to AV affecting the chest and/or back, a common presentation in acne patients.

Compounds with activity of retinoid type (vitamin A and its derivatives) are widely known for their potential in the treatment or prevention of dermatologic conditions. Several of the biological effects of retinoids are mediated by modulating the nuclear retinoic acid receptors (RAR), which activate transcription by binding to DNA sequence elements, known as RAR response elements (RARE), in the form of a heterodimer with the retinoid X receptors (known as RXRs). Three subtypes of human RARs have been identified and described: RARα, RARβ and RARγ. Trifarotene is a new selective retinoic acid receptor-γ (RARγ) topical retinoid and has unique selectivity for RARγ which distinguishes it from the existing first and third generation topical retinoids, which target both RARβ and RARγ. Trifarotene is metabolically stable in keratinocytes but rapidly metabolized in hepatic microsomes, predicting a favorable systemic safety profile; in addition, it has comedolytic, anti-inflammatory, and antipigmenting properties.

There remains a need to develop novel therapeutic regimes to treat patients with truncal skin conditions, particularly those suffering from truncal acne.

### SUMMARY

Provided herein are methods for treating acne vulgaris of the face and/or trunk in a subject, pharmaceutical compositions for use in the treatment of acne vulgaris of the face and/or trunk, uses of trifarotene or an equivalent thereof in the manufacture of a medicament for the treatment of acne vulgaris of the face and/or trunk.

In accordance with one or more embodiments, there are provided methods of treating acne vulgaris of the face and/or trunk in a subject, the method comprising, consisting of, or consisting essentially of administering a pharmaceutical composition comprising trifarotene or an equivalent thereof to the subject.

In one or more embodiments, the subject has acne vulgaris of the face and/or trunk. In one or more embodiments of the compositions, the subject has been diagnosed of acne vulgaris of the face and/or trunk. In one or more embodiments, the method comprises treatment of acne vulgaris of the trunk.

In one or more embodiments, trifarotene or the equivalent thereof is administered once daily, twice daily, once per week, once every two weeks, once every three weeks, once every four weeks, once every five weeks, once every six weeks, once every seven weeks, or once every eight weeks. In one or more embodiments, the administering comprises applying the pharmaceutical composition to the affected area of the subject. In one or more embodiments, trifarotene or the equivalent thereof is administered once daily to the affected areas of said subject. In one or more embodiments, containing from about 1 μg/g to about 100 μg/g of trifarotene or an equivalent thereof and a pharmaceutically acceptable carrier is topically administered to the subject. In one or more embodiments, a cream comprising about 50 μg/g of trifarotene or an equivalent thereof and a pharmaceutically acceptable carrier, is topically administered once daily to the affected areas of said subject.

In one or more embodiments, the amount of trifarotene or the equivalent thereof administered to the subject is about 0.00001 to about 1% by weight.

In one or more embodiments, the treatment reduces the inflammatory lesion counts of the subject. In one or more embodiments, the treatment reduces the noninflammatory lesion counts of the subject.

In one or more embodiments of the uses, the acne vulgaris of the face and/or trunk may include mild, moderate and severe. In one or more embodiments, the acne vulgaris of the face and/or trunk is moderate.

In accordance with one or more embodiments, there are provided pharmaceutical composition comprising trifarotene or an equivalent thereof and a pharmaceutically acceptable carrier.

In accordance with one or more embodiments, there are provided pharmaceutical compositions for use in the treatment of acne vulgaris of the face and/or trunk in a subject, the composition comprising, consisting of, or consisting essentially of trifarotene or an equivalent thereof.

In one or more embodiments, the pharmaceutical composition comprises trifarotene or an equivalent thereof and a carrier. In one or more embodiments, the carrier is a pharmaceutically acceptable carrier. In one or more embodiments, the pharmaceutically acceptable carrier comprises a liquid, paste or solid form, and more particularly in the form of ointments, creams, milks, pomades, powders, impregnated pads, syndets, solutions, gels, sprays, foams, pastes, suspensions, sticks, shampoos or washing bases. In one or more embodiments, the pharmaceutically acceptable carrier comprises suspensions of microspheres or nanospheres or of lipid or polymer vesicles or gelled or polymer patches which allow a controlled release. In one or more embodiments, the pharmaceutically acceptable carrier comprises one or more of an ointment, a cream, a gel, a spray, a foam, a paste or a suspension.

In one or more embodiments, the pharmaceutical composition is formulated as a cream. In one or more embodiments, the pharmaceutical composition is formulated as an oil-in-water emulsion. In one or more embodiments, the pharmaceutical composition comprises trifarotene or an equivalent thereof in an amount of from about 0.001 to about 0.1% by weight, relative to the weight of the total composition.

In accordance with one or more embodiments, there are provided uses of trifarotene or an equivalent thereof in the manufacture of a medicament for the treatment of acne vulgaris of the face and/or trunk in a subject.

### BRIEF DESCRIPTION OF THE DRAWINGS

FIG. 1 is a graph of the success rate in percentage, over time, comparing trifarotene (50 μg/g) cream and vehicle cream in the Intent-to-Treat (ITT) face acne vulgaris population for PERFECT 1 study.

FIG. 2 is a graph of the mean change in inflammatory lesion count, over time for trifarotene (50 μg/g) cream and vehicle cream, in the ITT face acne vulgaris population for PERFECT 1 study.

FIG. 3 is a graph of the mean change in non-inflammatory lesion count, over time for trifarotene (50 μg/g) cream and vehicle cream, in the ITT face acne vulgaris population for PERFECT 1 study.

FIG. 4 is a graph of the success rate in percentage, over time, comparing trifarotene (50 μg/g) cream and vehicle cream in the Intent-to-Treat (ITT) trunk acne vulgaris population for PERFECT 1 study.

FIG. 5 is a graph of the mean change in inflammatory lesion count, over time for trifarotene (50 μg/g) cream and vehicle cream, in the ITT trunk acne vulgaris population for PERFECT 1 study.

FIG. 6 is a graph of the mean change in non-inflammatory lesion count, over time for trifarotene (50 μg/g) cream and vehicle cream, in the ITT trunk acne vulgaris population for PERFECT 1 study.

FIG. 7 is a graph of the success rate in percentage, over time, comparing trifarotene (50 μg/g) cream and vehicle cream in the Intent-to-Treat (ITT) face acne vulgaris population for PERFECT 2 study.

FIG. 8 is a graph of the mean change in inflammatory lesion count, over time for trifarotene (50 μg/g) cream and vehicle cream, in the ITT face acne vulgaris population for PERFECT 2 study.

FIG. 9 is a graph of the mean change in non-inflammatory lesion count, over time for trifarotene (50 μg/g) cream and vehicle cream, in the ITT face acne vulgaris population for PERFECT 2 study.

FIG. 10 is a graph of the success rate in percentage, over time, comparing trifarotene (50 μg/g) cream and vehicle cream in the Intent-to-Treat (ITT) trunk acne vulgaris population for PERFECT 2 study.

FIG. 11 is a graph of the mean change in inflammatory lesion count, over time for trifarotene (50 μg/g) cream and vehicle cream, in the ITT trunk acne vulgaris population for PERFECT 2 study.

FIG. 12 is a graph of the mean change in non-inflammatory lesion count, over time for trifarotene (50 μg/g) cream and vehicle cream, in the ITT trunk acne vulgaris population for PERFECT 2 study.

FIGS. 13A-13D are graphs of acne vulgaris tolerability profile of trifarotene and vehicle from baseline to week 12, in the ITT face acne vulgaris population for PERFECT 1 study.

FIGS. 14A-13D are graphs of acne vulgaris tolerability profile of trifarotene and vehicle from baseline to week 12, in the ITT trunk acne vulgaris population for PERFECT 1 study.

FIGS. 15A-15D are graphs of acne vulgaris tolerability profile of trifarotene and vehicle from baseline to week 12, in the ITT face acne vulgaris population for PERFECT 2 study.

FIGS. 16A-16D are graphs of acne vulgaris tolerability profile of trifarotene and vehicle from baseline to week 12, in the ITT face acne vulgaris population for PERFECT 2 study.

FIG. 17 is a graph of the success rate in percentage, over time, comparing trifarotene (50 μg/g) cream and vehicle cream in the Intent-to-Treat (ITT) trunk acne vulgaris population for SATisFy study.

FIG. 18 is a graph of acne vulgaris tolerability profile of trifarotene from baseline to week 52, in the ITT face acne vulgaris population for SATisFy study.

FIG. 19 is a graph of acne vulgaris tolerability profile of trifarotene from baseline to week 52, in the ITT trunk acne vulgaris population for SATisFy study.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in one or more embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used herein, the singular forms “a,” “an,” and “the” designate both the singular and the plural, unless expressly stated to designate the singular only.

It is to be understood, although not always explicitly stated, that all numerical designations are preceded by the term “about.” The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 15%, 10%, 5%, 1%, or 0.1% of the particular term.

Also as used herein, “and/or” refers to and encompasses any and all possible combinations of one or more of the associated listed items, as well as the lack of combinations when interpreted in the alternative (“or”). Thus, acne vulgaris of the face and/or trunk includes acne vulgaris of the face; acne vulgaris of the trunk; or acne vulgaris of the face and trunk.

The terms “administer,” “administration,” or “administering” as used herein refer to (1) providing, giving, dosing and/or prescribing, such as by either a health professional or his or her authorized agent or under his direction, and (2) putting into, applying, taking or consuming, such as by a health professional or the subject. For example, administration can include without limitation, topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

The terms “treat”, “treating” or “treatment”, as used herein, include alleviating, abating or ameliorating acne vulgaris of the face and/or trunk, or one or more symptoms thereof, whether or not acne vulgaris of the face and/or trunk is considered to be “cured” or “healed” and whether or not all symptoms are resolved. The terms also include reducing or preventing progression of acne vulgaris of the face and/or trunk or one or more symptoms thereof, impeding or preventing an underlying mechanism of acne vulgaris of the face and/or trunk or one or more symptoms thereof, and achieving any therapeutic and/or prophylactic benefit.

As used herein, the term “subject” is used interchangeably with “patient,” and indicates a mammal, in particular a human, equine, bovine, porcine, feline, canine, murine, rat, or non-human primate. In one or more embodiments, the subject is a human.

As used herein, the term “equivalent thereof” as used herein, include, for example, salts, precursors, derivatives, esters, polymorphs, etc. of the therapeutic active agent. For example, an equivalent of trifarotene can include trifarotene salts.

The term “trifarotene salts” especially means the salts formed with a pharmaceutically acceptable base, such as an alkali metal or alkaline-earth metal salt, or alternatively a zinc salt or a salt of an organic amine or of an acidic partner when the compound is itself basic.

The term “precursors” means the immediate biological precursors or substrates thereof, and also the chemical precursors thereof.

The term “derivatives” means both the metabolic derivatives thereof and the chemical derivatives thereof.

An “effective amount” is an amount sufficient to effect beneficial or desired results such as alleviating at least one or more symptom of acne vulgaris of the face and/or trunk. An effective amount as used herein would also include an amount sufficient to delay the development of or alter the course of an acne vulgaris of the face and/or trunk and/or a symptom (for example low self-esteem), or reverse a symptom of acne vulgaris of the face and/or trunk. Thus, it is not possible to specify the exact “effective amount.” However, for any given case, an appropriate “effective amount” can be determined by one of ordinary skill in the art using only routine experimentation. For example, the effective amount of pharmaceutical composition of trifarotene may include the use of a thin layer of the composition sufficient to cover the area to be treated.

In one or more embodiments, acne vulgaris are scored as clear, almost clear, mild, moderate, or severe. “clear,” “almost clear,” “mild,” “moderate,” and “severe” are terms of art in describing the presence, extent, anatomical location, extent on the body, type of morphological lesions (e.g., papules, pustules, nodules, cysts, scars, comedones, etc.), severity, and/or intensity of the acne. Those of skill in the art know the metes and bounds of these terms.

The term “carrier” refers to a diluent, adjuvant, excipient, or vehicle with which the therapeutic agent is administered.

### Therapeutic Active Agent

Disclosed are pharmaceutical compositions, dosages and methods of treatment of facial and/or truncal acne in a subject, wherein the therapeutic active agent is a retinoid or an equivalent thereof. Provided herein are therapeutic agents for acne vulgaris of the face and trunk which comprise a retinoid. Suitable retinoids include those disclosed in PCT Publication No. WO 2006/066 978 such as 3″-tert-butyl-4′-(2-hydroxyethoxy)-4″-pyrrolidin-1-yl-[1,1′; 3′,1″]ter-phenyl-4-carboxylic acid, and oil/water-emulsion-type topical compositions containing such retinoids are disclosed in WO 2013/178760, the contents of each of these are relied upon and incorporated herein by reference in their entirety. In one or more embodiments, the retinoid is trifarotene. In one or more embodiments, the present disclosure provides therapeutic agents for acne vulgaris of the face and trunk which comprise trifarotene or an equivalent thereof as an active ingredient.

Trifarotene is a terphenyl acid derivative, that belongs to a new generation of the pharmacological class of retinoid acid receptor γ (RARγ) agonist. The chemical name of trifarotene is 3″-tert-Butyl-4′-(2-hydroxy-ethoxy)-4″-pyrrolidin-1-yl-[1,1′,3′,1″]terphenyl-4-carboxylic acid, and the molecular formula is C₂₉H₃₃NO₄ (molecular weight 459.58) and following structural formula:

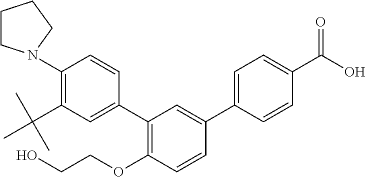 [SMILES: CC(C)(C)c1cc(-c2cc(-c3ccc(C(=O)O)cc3)ccc2OCCO)ccc1N1CCCC1]

Trifarotene is a first-in-class RARγ-selective topical retinoid cream formulation that is potent at low concentrations with a high safety margin and low systemic exposure, making it suitable for use on large body areas. Trifarotene 50 μg/g cream was developed for the treatment of moderate facial acne including large surface areas and is being assessed for its efficacy in treating truncal acne, including large surface areas.

Trifarotene is marketed under the trademark AKLIEF® at a concentration of 50 micrograms per gram (mcg/g or μg/g), in the form of a cream. These compositions are useful for treating acne vulgaris of the face and/or trunk.

### Pharmaceutical Compositions

Provided herein are pharmaceutical compositions for use in the treatment of acne vulgaris of the face and trunk, the composition comprising, consisting of, or consisting essentially of trifarotene or an equivalent thereof.

In one aspect, the present technology provides a pharmaceutical composition including trifarotene or an equivalent thereof as described herein and a pharmaceutically acceptable carrier. In one or more embodiments, the pharmaceutical composition may include a therapeutically effective amount of trifarotene or an equivalent thereof and a pharmaceutically acceptable carrier. In some embodiments, an effective amount is one that reduces the incidence and/or severity of, and/or delays onset of, one or more symptoms of the disease, disorder, and/or condition associated with acne vulgaris of the face and/or trunk. One of ordinary skill in the art will appreciate that the term “effective amount” may not require successful treatment be achieved in a particular individual. In some embodiments, effective amount may be that amount that provides a particular desired pharmacological response in a significant number of subjects when administered to patients in need of such treatment. In some embodiments, an effective amount may be formulated and/or administered in a single dose. In other embodiments, an effective amount may be formulated and/or administered in a plurality of doses, for example, as part of a dosing regimen. In one or more embodiments, the effective amount may be an effective amount for treating acne vulgaris, including acne vulgaris of the face and/or trunk disclosed herein.

It is recognized that pharmaceutical compositions comprising trifarotene or an equivalent thereof may be provided in the form of a cream or a foam which may be dispensed from a tube or a pump. In some embodiments, the pharmaceutical compositions may be packed as a 50 mL bottle with pump and over cap. In some embodiments, the pharmaceutical composition provided herein includes an effective amount of an active ingredient, which includes an amount (and/or a concentration) sufficient so that a single actuation of a pump containing the composition produces an effective amount. In some embodiments, more than one actuation of the pump may be required to produce an effective amount of active ingredient. For example, one pump actuation may include an amount of the pharmaceutical composition which is enough to cover the face (i.e., forehead, cheeks, nose, and chin), two actuations of the pump may include an amount of the pharmaceutical composition which is enough to cover the upper trunk (i.e., reachable upper back, shoulders and chest). In some embodiments, one additional pump actuation may be used for application to the middle and lower back if acne is present.

In one or more embodiments, the pharmaceutical compositions may contain trifarotene, or an equivalent thereof, in an amount corresponding to 0.00001% to 10% by weight, including, but not limited to, from about from about 0.00001 to 5% by weight, about 0.00001 to 2% by weight, about 0.00001 to 1% by weight, 0.00001 to 0.1% by weight, about 0.00005 to 10% by weight, about 0.00005 to 5% by weight, about 0.00005 to 2% by weight, about 0.00005 to 1% by weight, 0.00005 to 0.1% by weight, about 0.0001 to 10% by weight, about 0.0001 to 5% by weight, about 0.0001 to 2% by weight, about 0.0001 to 1% by weight, 0.0001 to 0.1% by weight, about 0.0005 to 10% by weight, about 0.0005 to 5% by weight, about 0.0005 to 2% by weight, or about 0.0005 to 1% by weight, about 0.0005 to 0.1% by weight, about 0.001 to 10% by weight, about 0.001 to 5% by weight, about 0.001 to 2% by weight, about 0.001 to 1% by weight, about 0.001 to 0.1% by weight, of trifarotene or an equivalent thereof, relative to the weight of the total composition. or any range including and/or in-between any two of these values. In one or more embodiments, the pharmaceutical compositions may contain trifarotene, or an equivalent thereof, in an amount of from about 0.001 to about 0.1% by weight, relative to the weight of the total composition, with pharmaceutically acceptable excipients and/or carrier constituting the remaining amount of the pharmaceutical composition.

The pharmaceutical compositions include trifarotene or an equivalent thereof as at least one of the active ingredients. In addition, the therapeutic agents for acne vulgaris of the face and/or trunk, in the present disclosure may also comprise, in combination with trifarotene or an equivalent thereof, other ingredients that enhance the treatment of acne vulgaris of the face and/or trunk. For example, the composition may comprise one or more topical creams, moisturizers, liquid, paste or solid form, and more particularly in the form of ointments, creams, milks, pomades, powders, impregnated pads, syndets, solutions, gels, sprays, foams, pastes, suspensions, sticks, shampoos or washing bases. It may also be in the form of suspensions of microspheres or nanospheres or of lipid or polymer vesicles or gelled or polymer patches allowing a controlled release. In some embodiments, the pharmaceutically acceptable carrier includes one or more of an ointment, a cream, a gel, a spray, a foam, a paste, or a suspension.

The pharmaceutical compositions of any embodiment herein may be formulated for topical administration or any of the routes discussed herein. In one or more embodiments, pharmaceutical compositions of trifarotene or an equivalent thereof may be formulated as a composition for topical administration. The pharmaceutical compositions of the present technology are particularly suited for topical treatment of the skin, and may be in the form of ointments, creams, milks, pomades, powders, impregnated pads, solutions, gels, gel-creams, sprays, lotions, foams or suspensions. For example, the pharmaceutical compositions may include a foam containing trifarotene compositions disclosed in PCT Publication No. WO 2013/178760, the contents of which are relied upon and incorporated herein by reference in their entirety. In one or more embodiments, the compositions may be in the form of suspensions of microspheres or nanospheres or of lipid or polymeric vesicles, or of polymeric patches and hydrogels for controlled release. These compositions for topical application may be in anhydrous form, in aqueous form or in the form of an emulsion. In one or more embodiments, the pharmaceutical composition of the present technology is in the form of a cream, a gel, a gel-cream or a lotion. In one or more embodiments, the pharmaceutical composition is formulated as a cream (e.g., face and/or truncal cream).

The pharmaceutical compositions described herein may contain various carriers or excipients known to those skilled in the art. Suitable carriers or excipients may include, but are not limited to, emollients, ointment base, emulsifying agents, solubilizing agents, humectants, thickening or gelling agents, wetting agents, texture enhancers, stabilizers, pH regulators, osmotic pressure modifiers, emulsifiers, UV-A and UV-B screening agents, preservatives, permeation enhancer, chelating agents, antioxidants, acidifying agents, alkalizing agents, buffering agents and vehicle or solvent. In one or more embodiments, the pharmaceutically acceptable carrier includes a liquid, paste or solid form, and more particularly in the form of ointments, creams, milks, pomades, powders, impregnated pads, syndets, solutions, gels, sprays, foams, pastes, suspensions, sticks, shampoos or washing bases. It may also be in the form of suspensions of microspheres or nanospheres or of lipid or polymer vesicles or gelled or polymer patches allowing a controlled release.

Besides those representative excipients described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Pharmaceutical compositions of trifarotene or an equivalent thereof of the present disclosure can be prepared as formulations according to standard methods which are also described in Remington's Pharmaceutical Science, Mark Publishing Co., New Jersey (1991).

The pharmaceutical compositions of the present disclosure are preferably administered topically. Specifically, the pharmaceutical compositions are administered to subjects or patients by topical application. While the compositions may not be primarily designed for oral, ophthalmic, or intravaginal use, other systemic or local administration methods are contemplated.

### Methods of Treatment

In accordance with one aspect, provided are methods of treating acne vulgaris of the face and/or trunk, the method comprising, consisting of, or consisting essentially of administering a pharmaceutical composition, which includes trifarotene or an equivalent thereof, to the subject. In one or more embodiments, the method includes topically applying a pharmaceutical composition, which includes trifarotene or an equivalent thereof, to the subject. In one or more embodiments, the method includes topically administering an effective amount of trifarotene or an equivalent thereof to the subject. In one or more embodiments, the trifarotene or the equivalent thereof is administered by applying a thin layer enough to cover the area being treated.

In one or more embodiments, severity of the acne is characterized according to one or more of the following methods known by those skilled in the art. For example, the acne severity can be evaluated using the 5-point Investigator's Global Assessment (IGA) scale for the face and Physician's Global Assessment (PGA) scale for the trunk. In the present technology, IGA scores range from 0 to 4, as shown in Table 1 below.

TABLE 1
Investigator's Global Assessment and
Physician's Global Assessment Scales

| | | |
|---|---|---|
| 0 | Clear | Clear skin with no inflammatory or non-inflammatory |
| | | lesions. |
| 1 | Almost | A few scattered comedones and a few small papules. |
| | Clear | |
| 2 | Mild | Easily recognizable; less than half the surface is |
| | | involved. |
| | | Some comedones and some papules and pustules. |
| 3 | Moderate | More than half of the surface is involved. Many |
| | | comedones, papules and pustules. One nodule may be |
| | | present. |
| 4 | Severe | Entire surface is involved. Covered with comedones, |
| | | numerous papules and pustules. Few nodules may be |
| | | present. |

In one or more embodiments, the acne vulgaris is mild to moderate. In one or more embodiments, the acne vulgaris is moderate to severe. In one or more embodiments, the acne vulgaris of the face and/or trunk is moderate.

In one or more embodiments, the success rate of the treatment disclosed herein may be based on the IGA and PGA outcome (percentage of subjects “clear” and “almost clear” and with at least a 2-grade change from baseline) and/or the absolute and percentage change from baseline lesion counts on the face and/or trunk. The lesion counts consider the two major types of acne lesions: non-inflammatory and inflammatory. Non-inflammatory lesions of acne are the open (blackheads) or closed (whiteheads) comedones. Inflammatory lesions are divided into papules, pustules, and nodules/nodulocystic lesions, depending on the severity and location of the inflammation within the dermis. Both the absolute and percentage reduction in total number of inflammatory and/or non-inflammatory lesions on the face and/or trunk from the first day of treatment (baseline) to the end of the treatment may be used to assess the success rate.

In particular embodiments, the subject has at least 20 inflammatory lesions and 25 non-inflammatory lesion counts on the face at screening and baseline. In particular embodiments, the subject has at least 20 inflammatory lesions and 20 non-inflammatory lesions but no more than 100 non-inflammatory lesion counts on the trunk (shoulders, upper back and chest, anterior chest) at screening and baseline. In one or more embodiments, the treatment may include about ≥10% reduction in inflammatory and/or non-inflammatory lesion count on the face and/or trunk in a subject, including, but not limited to, greater than about 15%, greater than about 20%, greater than about 30%, greater than about 40% or greater than about 50% reduction in inflammatory and/or non-inflammatory lesion count. In one or more embodiment, the treatment may include from about 5% to about 99% reduction in inflammatory and/or non-inflammatory lesion count in a subject, including, but not limited to, about 10% to about 90%, about 15% to about 85%, about 20% to about 80%, about 25% to about 75%, about 30% to about 70%, about 35% to about 65%, about 40% to about 60%, about 45% to about 50% reduction in inflammatory and/or non-inflammatory lesion count on the face and/or trunk, or any range including and/or in-between any two of these values. In one or more embodiments, the treatment includes about 20% to about 60% reduction in inflammatory and/or non-inflammatory lesion count on the face and/or trunk after 12 weeks of treatment. In one or more embodiments, the treatment includes about 30% to about 50% reduction in inflammatory and/or non-inflammatory lesion count on the face and/or trunk after 12 weeks of treatment.

In one or more embodiments, the subject has acne vulgaris of the face and trunk, including moderate acne vulgaris of the face and trunk. In one or more embodiments, the subject has been diagnosed of having acne vulgaris of the face and trunk with a severity grade of 3 (moderate) on the IGA scale at screening and baseline visits or a truncal acne severity grade of 3 (moderate) on the PGA scale at screening and baseline visits on trunk (shoulders, upper back and anterior chest). In particular embodiments, the subject has at a minimum of 20 inflammatory lesions and 20 non-inflammatory lesions but no more than 100 non-inflammatory lesion counts on the trunk. In particular embodiments, the acne or lesions are reachable to self-application of study drug by the subject at Screening and Baseline (optional criterion for subject between 9 and 11 years of age).

An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, skin absorption for topical compositions, systemic absorption in general for topical and oral compositions, and the severity of the particular disorder being treated and form of administration. Treatment dosages generally may be titrated to optimize safety and efficacy. The dosage can be determined by a physician and adjusted, as necessary, to suit observed treatment effects and to manage and adapt to retinoid dermatitis.

In one or more embodiments, creams, ointments, foams or lotions which includes from about 1 μg/g to about 100 μg/g of trifarotene or an equivalent thereof is topically administered to the subject, including, but not limited to, from about 5 μg/g to about 95 μg/g, about 10 μg/g to about 90 μg/g, about 15 μg/g to about 85 μg/g, about 20 μg/g to about 80 μg/g, about 25 μg/g to about 75 μg/g, about 30 μg/g to about 70 μg/g, about 35 μg/g to about 65 μg/g, about 40 μg/g to about 60 μg/g, about 45 μg/g to about 55 μg/g, about 46 μg/g to about 54 μg/g, about 47 μg/g to about 53 μg/g, about 48 μg/g to about 52 μg/g, or about 49 μg/g to about 51 μg/g. In certain embodiments, a cream which includes from about 1 μg/g to about 100 μg/g of trifarotene or an equivalent thereof is topically administered to the subject. In certain embodiments, a cream which includes about 50 μg/g of trifarotene or an equivalent thereof is topically administered once daily to the affected area of the subject.

In one or more embodiments, the effective amount of trifarotene or the equivalent thereof ranges from about 0.0001 weight percent to about 0.1 weight percent, about 0.001 weight percent to about 0.5 weight percent, about 0.005 weight percent to about 1 weight percent, about 0.01 weight percent to about 1.5 weight percent, or about 0.1 weight percent to about 10 weight percent. In particular embodiments, the effective amount of trifarotene or the equivalent thereof is about 0.0001 weight percent, about 0.005 weight percent, about 0.1 weight percent, about 1 weight percent, about 2 weight percent, or about 2.5 weight percent, or any range including and/or in-between any two of these values, and/or as needed based on the appearance of symptoms of acne vulgaris of the face and/or trunk. In particular embodiments, the effective amount of trifarotene or the equivalent thereof is a 2 g flat dose (e.g. in cream form).

In one or more embodiments, the trifarotene or the equivalent thereof is administered by a topical route. In one or more embodiments, the trifarotene or the equivalent thereof is administered by applying a thin layer enough to cover the area being treated. In one or more embodiments, the dose is administered proximal to a site of one or more nodules. In one or more embodiments, trifarotene or the equivalent thereof, in a topical form such as cream, is applied as a thin layer to the face, and/or to the chest, shoulders, and back. In some embodiments, the subject may choose to avoid applying trifarotene or the equivalent thereof to damaged skin (such as cuts, abrasions), to axillary areas, neck, eczematous areas, and sunburned skin. In one or more embodiments, the trifarotene or the equivalent thereof is topically administered to the affected area of the subject. In one or more embodiments, the trifarotene or the equivalent thereof is administered by applying a thin layer of the pharmaceutical composition containing trifarotene or the equivalent thereof to the affected areas of the face and/or trunk once a day, in the evening, on clean and dry skin.

In one or more embodiments, trifarotene or the equivalent thereof is administered thrice daily, twice daily, once daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, or any range including and/or in-between any two of these values, and/or as needed based on the appearance of symptoms of acne vulgaris of the face and/or trunk. In one or more embodiments, trifarotene or the equivalent thereof is administered once daily.

The treatments have a variable duration, depending on the patient and the severity of the facial and/or truncal acne. The treatment period may thus run from several days to several years. In one or more embodiments, the duration of treatment is about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about one week, about 2 weeks, about 3 weeks, about 4 weeks, about 5 weeks, about 10 weeks, about 20 weeks, about 30 weeks, about 36 weeks, about 40 weeks, about 48 weeks, about 50 weeks, about one year, about two years, about three years, about four years, about five years, or any range including and/or in-between any two of these values, and/or as needed based on the appearance of symptoms of acne vulgaris of the face and/or trunk. In one or more embodiments, duration of treatment is about 10 days to about 35 days, about 25 days to about 30 days, about 12 to about 48 weeks, or about 24 to about 36 weeks. Trifarotene or an equivalent thereof is safe and has surprisingly improved efficacy for the treatment of acne vulgaris of the face and/or trunk in a subject for a treatment duration of about 52 weeks or more. In one or more embodiments, pharmaceutical compositions of trifarotene or an equivalent thereof exhibit long term safety and efficacy in the treatment of moderate facial and truncal acne.

In one or more embodiments, provided are uses of trifarotene or an equivalent thereof in the manufacture of a medicament for the treatment of acne vulgaris of the face and/or trunk in a subject. In one or more embodiments, provided are pharmaceutical composition comprising a comprising an effective amount of trifarotene or an equivalent thereof and a pharmaceutically acceptable carrier, for use in a method of treating acne vulgaris of face and/or trunk in a subject.

### EXAMPLES

Various embodiments will be further clarified by the following examples, which are in no way intended to limit this disclosure thereto.

### Abbreviations Used

ANCOVA Analysis of covariance
AV Acne vulgaris
BPO Benzoyl peroxide
IGA Investigator global assessment (facial acne)
MI Multiple imputation
PGA Physician global assessment (truncal acne)
SD Standard deviation
TEAE Treatment emergent adverse event

### Example 1

### Determination of Efficacy of Trifarotene in the Treatment of Facial and Truncal Acne Patients

A multi-center (United States, Canada, Europe and Russia), 12-week, double-blinded, and vehicle controlled group study with a 1:1 randomization pattern (once daily trifarotene 50 μg/g or vehicle cream) was conducted in subjects with moderate facial and truncal acne. Enrolled patients or caregivers received instruction about how to apply a thin layer of study drug daily at bedtime on the face and self-reachable trunk. Patients were also instructed to cleanse the skin and not to apply moisturizer one hour before or one hour after study drug application. Use of moisturizer was encouraged from the initiation of treatment. Investigators could reduce the frequency of application to alternate days for a maximum of 2 weeks in the first 4 weeks following the Baseline visit when needed to manage irritation. The patients were selected based on the following criteria.

Inclusion Criteria:
1. Male or female patients 9 years or older.
2. Moderate facial acne, defined as: Investigator Global Assessment (IGA) score of 3 on the face. Minimum of 20 inflammatory lesions and 25 non-inflammatory lesions on the face at Screening and Baseline
3. Moderate truncal acne with Physician Global Assessment (PGA) score of 3 at Screening and Baseline. Minimum of 20 inflammatory lesions and 20 non-inflammatory lesions but no more than 100 non-inflammatory lesion counts on the trunk (shoulders, upper back and upper anterior chest) reachable to self-application of study drug by the subject at Screening and Baseline (optional criterion for subject between 9 and 11 years of age).
4. Accessible treated areas of the trunk were defined for the study. The trunk anatomical region assessment was pre-defined by a size-fitted T-shirt (patent # US00D7580485, Jun. 7, 2016). This ensured consistency for study assessment areas throughout the study duration.
5. For subjects aged 9 to 11 years, inclusion criteria regarding truncal acne were optional due to relative rarity of trunk involvement (compared with face) in this age group.

Exclusion Criteria:

Patients with the following conditions are excluded.
1. Severe forms of acne, >1 nodule on face, >1 nodule on trunk.
2. Presence of acne cysts, beard or facial hair that could interfere with study assessments.
3. Presence of tattoos that could interfere with study assessments.
4. Uncontrolled or serious disease/medical condition.
5. Clinically significant abnormal laboratory values.
6. Known or suspected allergies or sensitivities to planned study drugs.
7. Lactation or intent to conceive during the study in women.

### Forbidden Therapies:

Prohibited medication use and washout periods of 1-4 weeks were specified for use of anti-acne treatments (prescription and over-the-counter), nonsteroidal anti-inflammatory drugs, corticosteroids, antibiotics, and 6 months for oral retinoids and immunomodulators.

### Efficacy and Safety Assessments:

Primary Efficacy Endpoints
Success rate, defined as the percentage of subjects who achieved an IGA score on the face of 1 (Almost Clear) or 0 (Clear) and at least a 2-grade improvement from Baseline to Week 12.
Absolute and percent change (supportive efficacy endpoint) in facial inflammatory lesion count from Baseline to Week 12.
Absolute and percent change (supportive efficacy endpoint) in facial non-inflammatory lesion count from Baseline to Week 12.

Secondary Efficacy Endpoints
Success rate, defined as the percentage of subjects who achieved an PGA (trunk) score of 1 (Almost Clear) or 0 (Clear) and at least a 2-grade improvement from Baseline to Week 12.
Absolute and percent change (supportive efficacy endpoint) in truncal inflammatory lesion count from Baseline to Week 12.
Absolute and percent change (supportive efficacy endpoint) in truncal non-inflammatory lesion count from Baseline to Week 12.

Severity definitions for IGA and PGA scales [5-point scales ranging from 0 (clear) to 4 (severe)] were the same. Lesion counts were performed and IGA and PGA were assessed at Screening, Baseline, and Weeks 1, 2, 4, 8, and 12/ET visits.

Safety was assessed through adverse events, vital signs and routine laboratory testing. Safety assessments included treatment emergent adverse events (TEAE5), standard laboratory safety tests at screening and last study visit including hematology, blood chemistry, and urinalysis (Barc Central Laboratory, US and Belgium), physical examination, and monitoring of vital signs. Local tolerability signs/symptoms expected with a topical retinoid were collected separately from adverse events to better characterize the tolerability profile of trifarotene cream. These included erythema, scaling, dryness, and stinging/burning scored on a 4-point scale, none=0, mild=1, moderate=2, severe=3. Subjects were specifically queried at each study visit, including Baseline, for the presence of local signs or symptoms. Signs and/or symptoms of local cutaneous irritation assessed with the tolerability scale were considered to be Adverse Events if they were severe enough to lead to permanent discontinuation of study product treatment or required use of concomitant treatment including over-the-counter products (other than moisturizers). Subjects were assessed at screening/baseline and Weeks 1, 2, 4, 8 and 12.

### Treatment:

Trifarotene or the vehicle were applied once daily in the evening for 12 weeks. The liberal use of a non-comedogenic and hypoallergenic moisturizer and non-comedogenic sunscreen was allowed. Trifarotene or the vehicle were not to be applied to cuts, abrasions, eczematous, or sunburned skin.

### Results

### Subject Disposition, Demographics, and Baseline Characteristics

In PERFECT 1 study, 1,524 subjects were screened and 1,208 randomized; among randomized subjects, 612 received trifarotene cream and 596 vehicle. Discontinuations were 72 in trifarotene arm and 61 in vehicle arm, and in both cases were primarily due to withdrawal by subject and lost to follow up. In PERFECT 2 study, 1,293 subjects were screened and 1,212 randomized; among randomized subjects, 602 received trifarotene cream and 610 vehicle. Discontinuations were 44 in trifarotene arm and 37 in vehicle arm, and in both cases were primarily due to withdrawal by subject and lost to follow up. Demographic and baseline data are presented in Table 2. Treatment groups were similar, with an even distribution of pediatric and adult subjects, no significant gender differences, primarily Caucasian subjects with skin phototypes I-III.

TABLE 2
Patient Characteristics

| Perfect 1 | | | Perfect 2 | | |
|---|---|---|---|---|---|
| Trifarotene | | | Trifarotene | | |
| 50 μg/g | Vehicle | Overall | 50 ug/g | Vehicle | Overall |
| (N = 612) | (N = 596) | (N = 1208) | (N = 602) | (N = 610) | (N = 1212) |

Age (years)

| | | | | | | |
|---|---|---|---|---|---|---|
| n | 612 | 596 | 1208 | 602 | 610 | 1212 |
| Mean (SD) | 19.6 ± 6.88 | 19.3 ± 5.89 | 19.4 ± 6.41 | 19.6 ± 6.2 | 19.9 ± 6.4 | 19.7 ± 6.3 |
| Median | 17 | 18 | 18 | 18 | 18 | 18 |
| (Min, Max) | (9, 58) | (10, 50) | (9, 58) | (11.0, 49.0) | (11.0, 46.0) | (11.0, 49.0) |

Age Categories, n (%)

| | | | | | | | | | | | | |
|---|---|---|---|---|---|---|---|---|---|---|---|---|
| <18 years | 314 | (51.3) | 278 | (46.6) | 592 | (49.0) | 276 | (45.8) | 294 | (48.2) | 570 | (47.0) |
| 18 years | 298 | (48.7) | 318 | (53.4) | 616 | (51.0) | 326 | (54.2) | 316 | (51.8) | 642 | (53.0) |

Gender, n (%)

| | | | | | | | | | | | | |
|---|---|---|---|---|---|---|---|---|---|---|---|---|
| Male | 307 | (50.2) | 272 | (45.6) | 579 | (47.9) | 245 | (40.7) | 272 | (44.6) | 517 | (42.7) |
| Female | 305 | (49.8) | 324 | (54.4) | 629 | (52.1) | 357 | (59.3) | 338 | (55.4) | 695 | (57.3) |

Race, n (%)

| | | | | | | | | | | | | |
|---|---|---|---|---|---|---|---|---|---|---|---|---|
| White | 508 | (83.0) | 484 | (81.2) | 992 | (82.1) | 565 | (93.9) | 554 | (90.8) | 1119 | (92.3) |
| Black or African | 47 | (7.7) | 49 | (8.2) | 96 | (7.9) | 27 | (4.5) | 42 | (6.9) | 69 | (5.7) |
| American | | | | | | | | | | | | |
| Asian | 23 | (3.8) | 32 | (5.4) | 55 | (4.6) | 2 | (0.3) | 6 | (1.0) | 8 | (0.7) |
| American Indian or | 11 | (1.8) | 5 | (0.8) | 16 | (1.3) | 1 | (0.2) | 2 | (0.3) | 3 | (0.2) |
| Alaska Native | | | | | | | | | | | | |
| Native Hawaiian or | 1 | (0.2) | 1 | (0.2) | 2 | (0.2) | 0 | (0.0) | 1 | (0.2) | 1 | (0.1) |
| Other Pacific Islander | | | | | | | | | | | | |
| Multiple | 8 | (1.3) | 10 | (1.7) | 18 | (1.5) | 2 | (0.3) | 2 | (0.3) | 4 | (0.3) |
| Other | 14 | (2.3) | 15 | (2.5) | 29 | (2.4) | 5 | (0.8) | 3 | (0.5) | 8 | (0.7) |

Ethnicity, n (%)

| | | | | | | | | | | | | |
|---|---|---|---|---|---|---|---|---|---|---|---|---|
| Hispanic or Latino | 135 | (22.1) | 148 | (24.8) | 283 | (23.4) | 60 | (10.0) | 62 | (10.2) | 122 | (10.1) |
| Not Hispanic or Latino | 477 | (77.9) | 448 | (75.2) | 925 | (76.6) | 542 | (90.0) | 548 | (89.8) | 1090 | (89.9) |

Skin Phototype, n (%)

| | | | | | | | | | | | | |
|---|---|---|---|---|---|---|---|---|---|---|---|---|
| Type I | 31 | (5.1) | 34 | (5.7) | 65 | (5.4) | 36 | (6.0) | 37 | (6.1) | 73 | (6.0) |
| Type II | 197 | (32.2) | 182 | (30.5) | 379 | (31.4) | 274 | (45.5) | 249 | (40.8) | 523 | (43.2) |
| Type III | 233 | (38.1) | 227 | (38.1) | 460 | (38.1) | 233 | (38.7) | 248 | (40.7) | 481 | (39.7) |
| Type IV | 97 | (15.8) | 91 | (15.3) | 188 | (15.6) | 33 | (5.5) | 38 | (6.2) | 71 | (5.9) |
| Type V | 43 | (7.0) | 48 | (8.1) | 91 | (7.5) | 14 | (2.3) | 19 | (3.1) | 33 | (2.7) |
| Type VI | 11 | (1.8) | 14 | (2.3) | 25 | (2.1) | 12 | (2.0) | 19 | (3.1) | 31 | (2.6) |

Baseline Disease Characteristics, mean ± SD

| | | | | | | |
|---|---|---|---|---|---|---|
| Facial Inflammatory | 34.7 ± 13.02 | 34.8 ± 13.612 | 34.8 ± 13.31 | 36.1 ± 12.47 | 37.1 ± 15.06 | 36.6 ± 13.84 |
| Lesions | | | | | | |
| Facial Non-inflammatory | 54.0 ± 28.55 | 52.8 ± 26.08 | 53.4 ± 27.35 | 50.6 ± 25.93 | 51.2 ± 25.75 | 50.9 ± 25.83 |
| Lesions | | | | | | |
| Trunk Inflammatory | 36.9 ± 17.89 | 35.6 ± 16.70 | 35.3 ± 17.32 | 39.0 ± 16.16 | 39.1 ± 17.41 | 39.1 ± 16.80 |
| Lesions | | | | | | |
| Trunk Non-inflammatory | 46.4 ± 21.57 | 47.5 ± 21.94 | 46.9 ± 21.75 | 46.1 ± 20.17 | 45.7 ± 19.58 | 45.9 ± 19.87 |
| Lesions | | | | | | |

### Efficacy

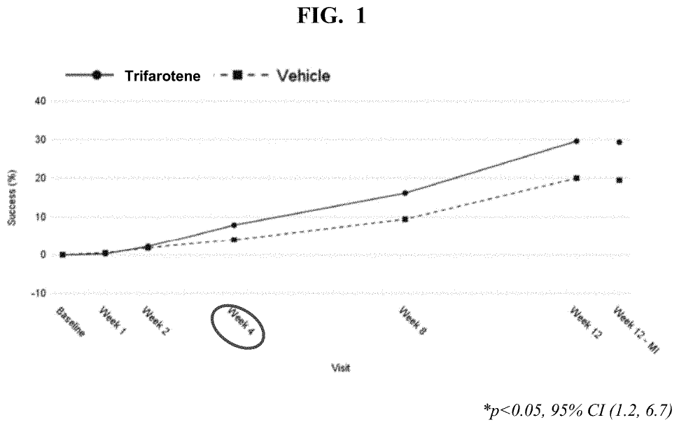

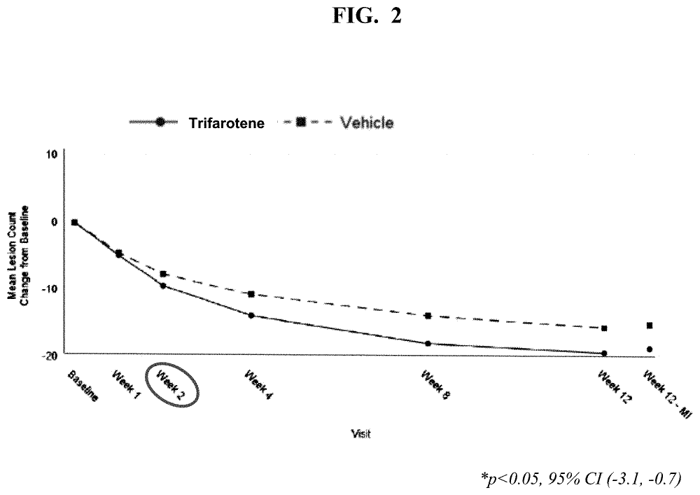

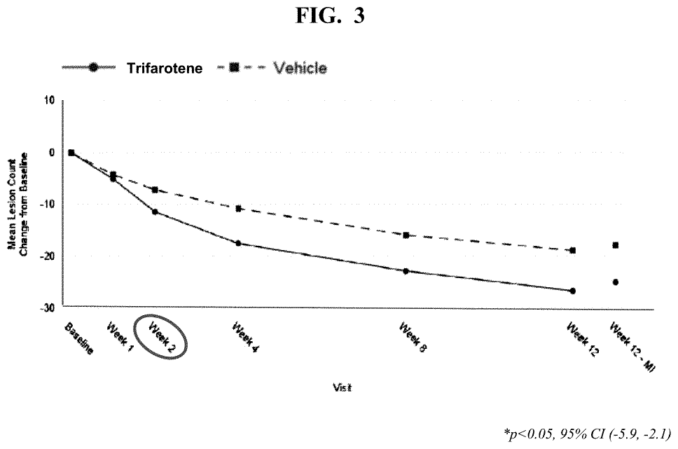

All co-primary and co-secondary efficacy assessments in both studies at Week 12 were statistically significant (p<0.001) in favor of trifarotene vs vehicle (Table 3). Among 1,214 patients treated with trifarotene and 1,206 treated with vehicle, week 12 facial IGA success rates were 29.4% in PERFECT 1 and 42.3% in PERFECT 2 (vs 19.5% and 25.7% for vehicle, P<0.001); trifarotene had statistically significant superior success rates at week 4 (PERFECT 1) and week 8 (PERFECT 2). Trifarotene treatment achieved significantly superior reductions in facial lesion counts as well, with statistical differences apparent as early as week 1-2: mean absolute inflammatory lesion count reductions were −19.0 and −24.2 (vs −15.4 and −18.7 vehicle, P<0.001); and mean absolute non-inflammatory lesion count reductions of —25.0 and −30.1 (vs −17.9 and −21.6 with vehicle, P<0.001). The three co-primary endpoints from baseline to Week 12 are shown in FIGS. 1-3 for PERFECT 1 study and 7-9 for PERFECT 2 study. Circles on the axis represent the period of early onset of effects.

TABLE 3
Summary efficacy results

| | PERFECT 1 | | PERFECT 2 | |
|---|---|---|---|---|
| | Trifarotene | Vehicle | Trifarotene | Vehicle |
| FACE: CO-PRIMARY | Cream | Cream | Cream | Cream |
| ENDPOINTS | (N = 612) | (N = 596) | (N = 602) | (N = 610) |
| IGA Success (%) | 29.4%^{a} | 19.5% | 42.3%^{a} | 25.7% |
| Inflammatory Lesions | | | | |
| Mean Absolute Change | −19.0^{a} | −15.4 | −24.2^{a} | −18.7 |
| from baseline | | | | |
| Mean % Change from | −54.4%^{a} | −44.8% | −66.2%^{a} | −51.2% |
| baseline | | | | |
| Non-inflammatory | | | | |
| Lesions | | | | |
| Mean Absolute Change | −25.0^{a} | −17.9 | −30.1^{a} | −21.6 |
| from baseline | | | | |
| Mean % Change from | −49.7%^{a} | −35.7% | −57.7%^{a} | −43.9% |
| baseline | | | | |
| | PERFECT 1 | | PERFECT 2 | |
| | Trifarotene | Vehicle | Trifarotene | Vehicle |
| TRUNK: SECONDARY | Cream | Cream | Cream | Cream |
| ENDPOINTS | (N = 600) | (N = 585) | (N = 598) | (N = 609) |
| PGA Success (%) | 35.7%^{a} | 25.0% | 42.6%^{a} | 29.9% |
| Inflammatory Lesions | | | | |
| Mean Absolute Change | −21.4^{a} | −18.8 | −25.5^{a} | −19.8 |
| from baseline | | | | |
| Mean Percent Change | −57.4%^{a} | −50.0% | −65.4%^{a} | −51.1% |
| from baseline | | | | |
| Non-inflammatory | | | | |
| Lesions | | | | |
| Mean Absolute Change | −21.9^{a} | −17.8 | −25.9^{a} | −20.8 |
| from baseline | | | | |
| Mean Percent Change | −49.1%^{a} | −40.3% | −55.2%^{a} | −45.1% |
| from baseline | | | | |

^{a}p < 0.001 vs. vehicle cream

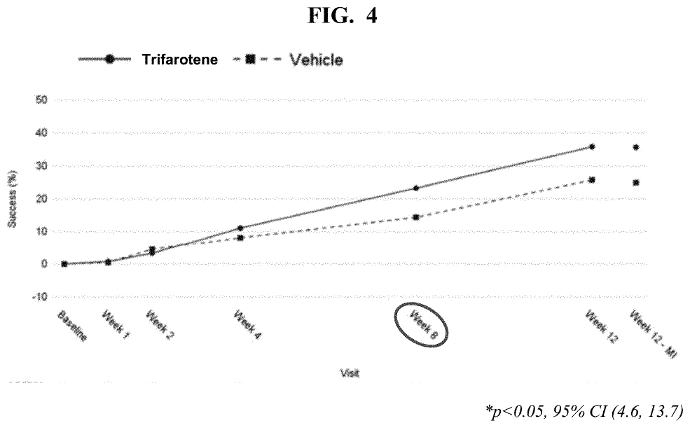

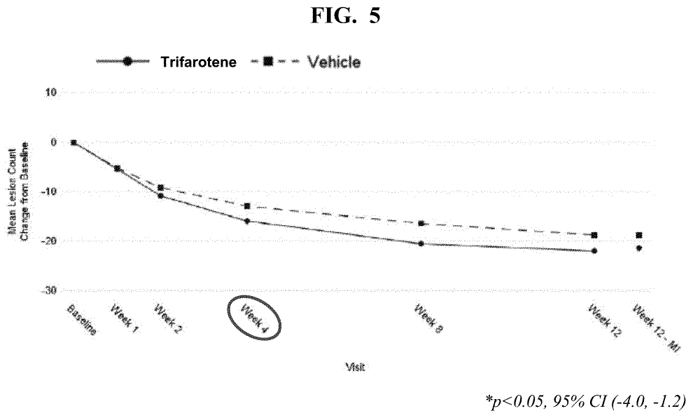

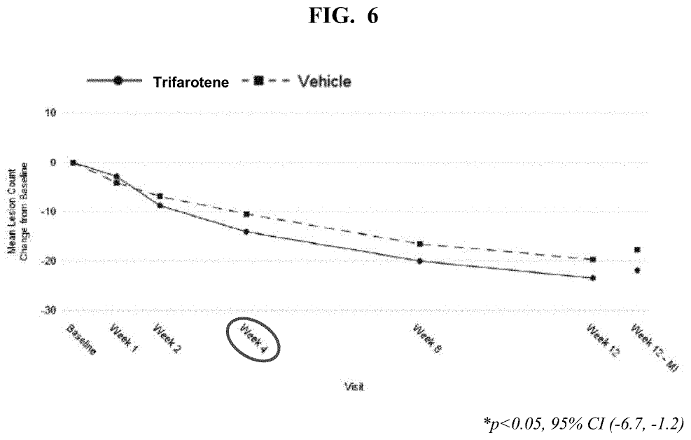

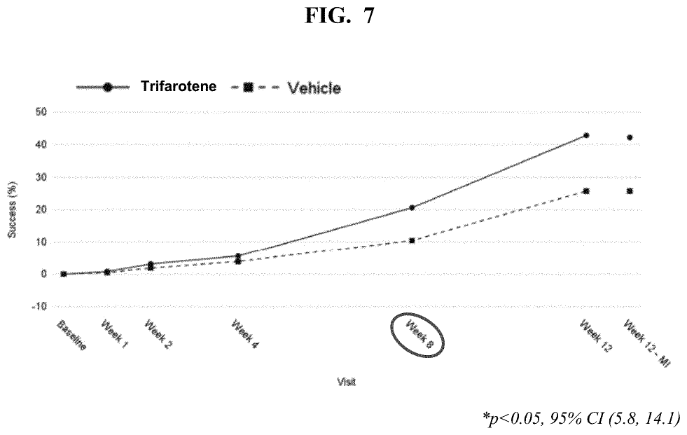

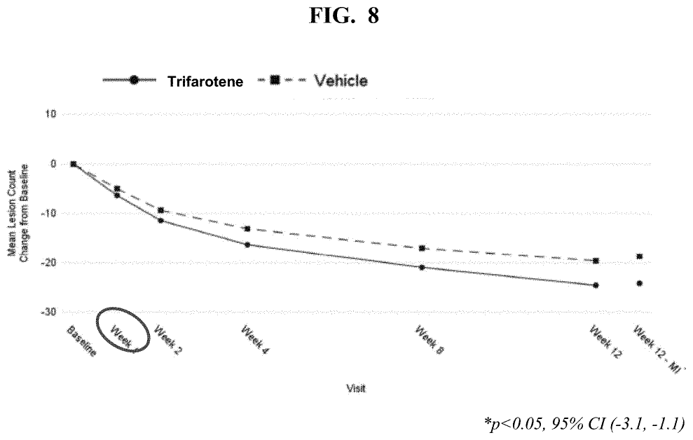

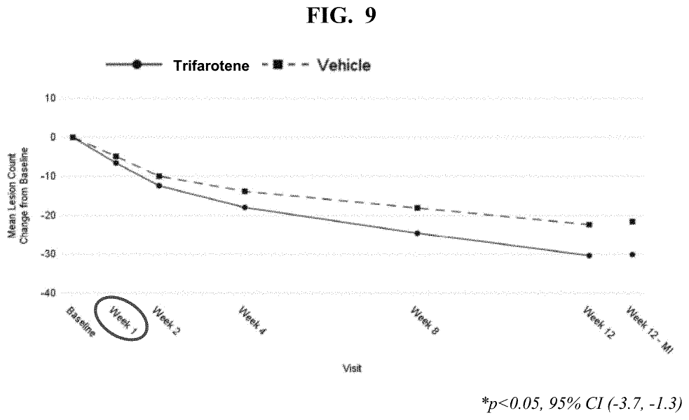

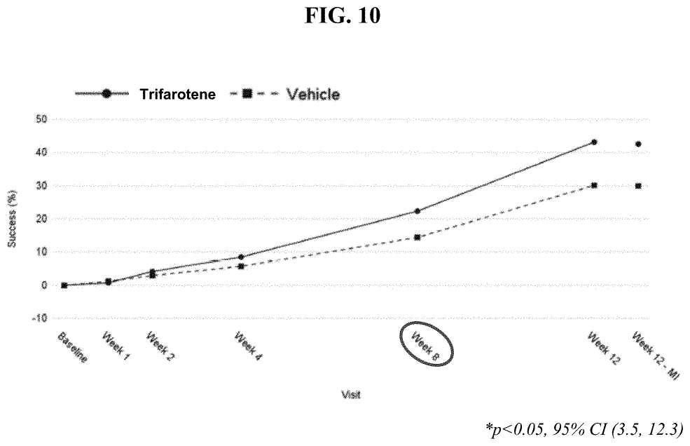

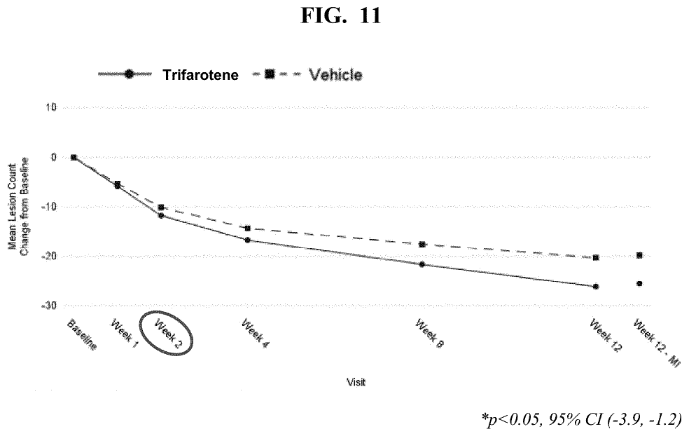

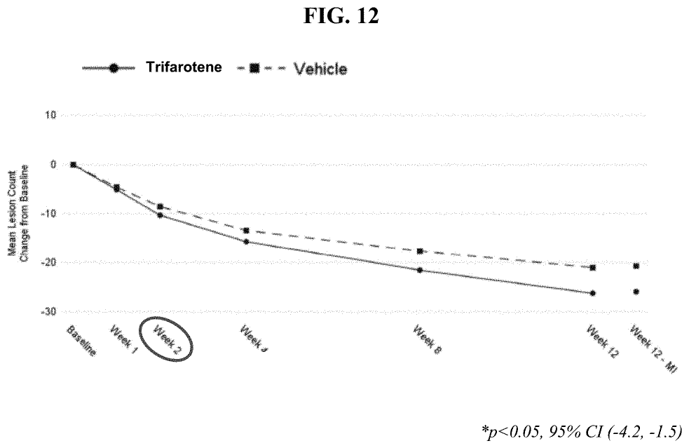

Secondary endpoints showing the treatment effect on truncal acne from baseline to Week 12 are presented in FIGS. 4-6 for PERFECT 1 study and FIGS. 10-12 for PERFECT 2 study. Circles represent the period of early onset of effects. At week 12, truncal PGA success rates with trifarotene were 35.7% in PERFECT 1 and 42.6% in PERFECT 2 (vs 25.0% and 29.9%, respectively for vehicle, each P<0.001). Trifarotene was also efficacious in reducing lesion counts on the trunk by week 8. In both PERFECT 1 and 2, trifarotene was statistically significantly superior in achieving reductions in inflammatory and non-inflammatory lesions on the trunk starting by week 4 in PERFECT 1 and week 2 in PERFECT 2. Success rates on the trunk were statistically significant for trifarotene vs vehicle starting at week 8 in both studies.

### Safety

### Local Tolerability

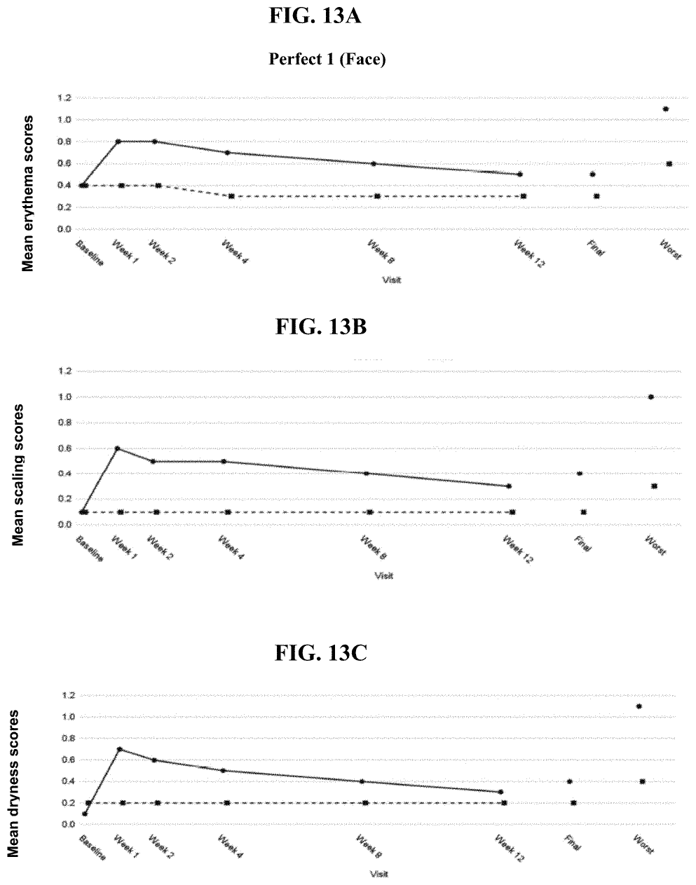

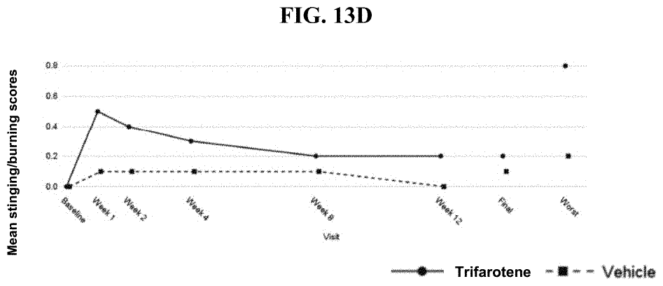

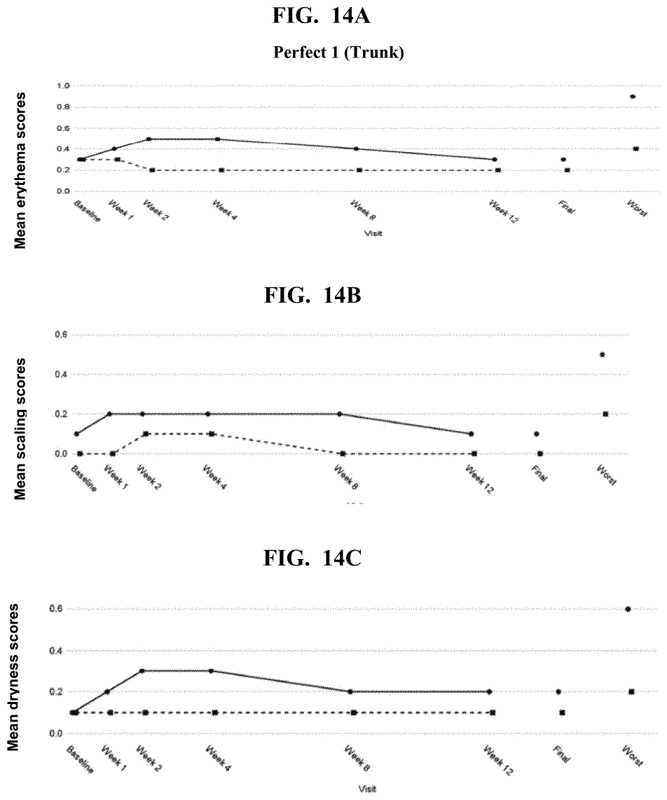

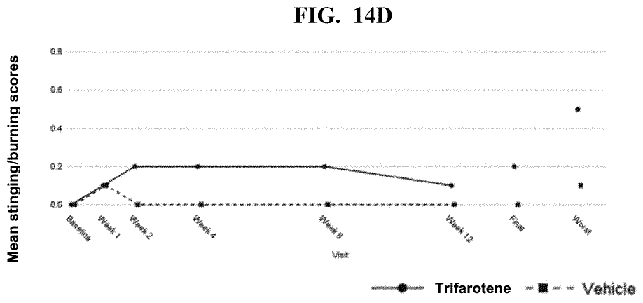

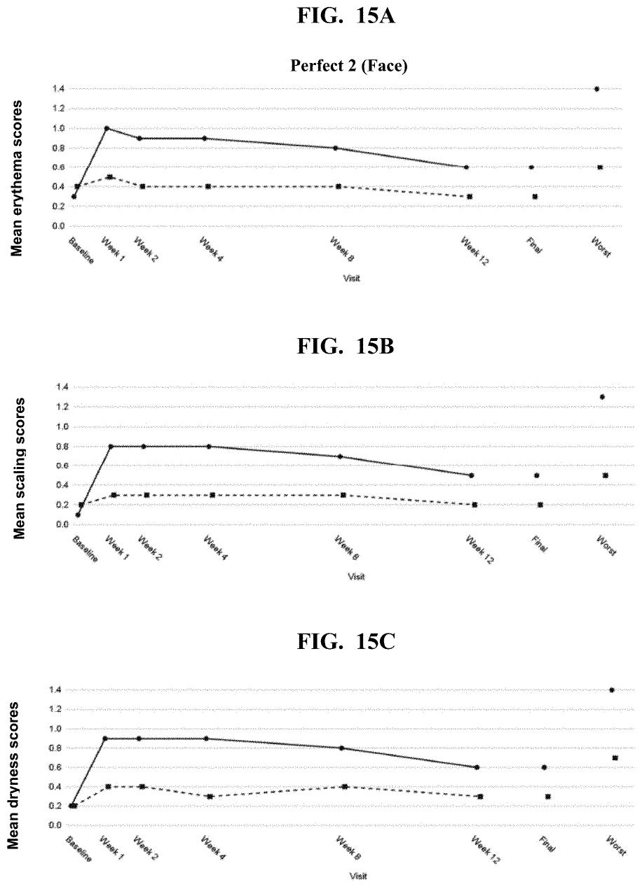

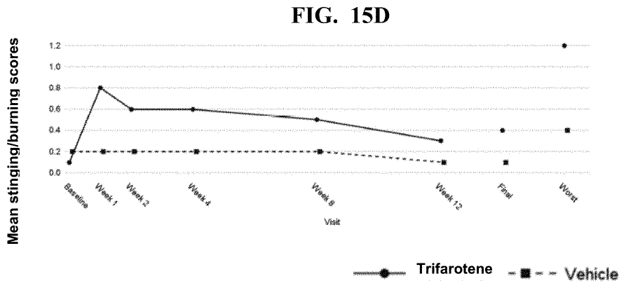

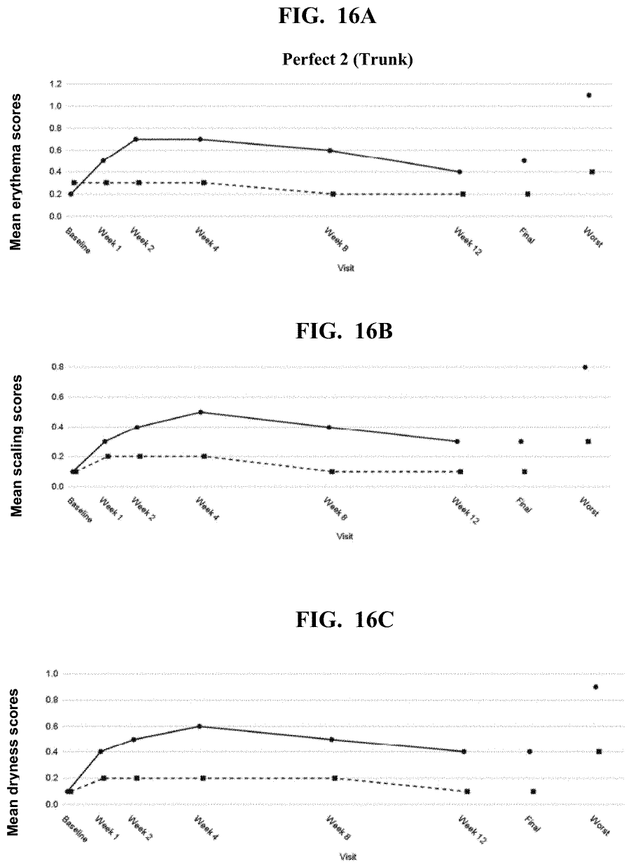

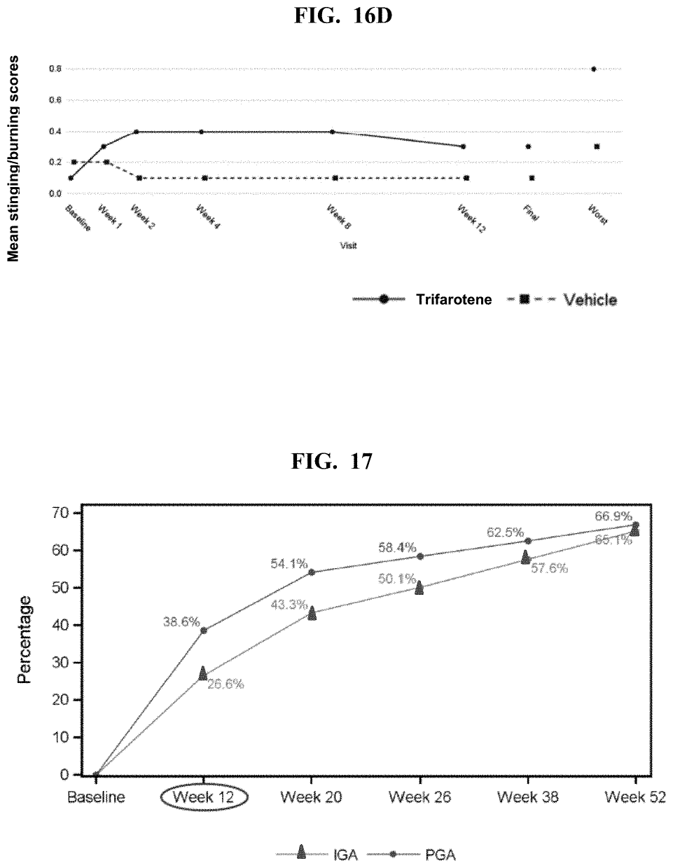

Local irritation related to trifarotene cream was transient and consistent with the known pattern of topical retinoid dermatitis (FIGS. 13-16; Severity scale score: 0 (none), 1 (mild), 2 (moderate), 3 (severe); tolerability was better on the trunk compared to the face. Local tolerability signs and symptoms related to trifarotene cream included erythema, scaling, dryness and stinging/burning. These were mostly mild to moderate by investigator assessment, with few being severe. For facial acne, a worst post-baseline score of moderate local tolerability signs/symptoms compared to baseline was reported for up to 33.2% of patients (PERFECT 1: erythema, 23.7%; scaling, 21.4%; dryness, 23.0%; stinging/burning, 16.3%. PERFECT 2: erythema, 33.2%; scaling, 32.9%; dryness, 36.4%; stinging/burning, 24.9%) and severe for up to 10.0% of patients (PERFECT 1: erythema, 2.5%; scaling, 2.9%; dryness, 2.5%; stinging/burning, 4.2%. PERFECT 2: erythema, 10.0%; scaling, 6.8%; dryness, 7.1%; stinging/burning, 7.6%). On the trunk, the corresponding percentages of worst post-baseline local tolerability signs/symptoms were moderate 23.2% (PERFECT 1: erythema, 14.6%; scaling, 10.8%; dryness, 11.3%; stinging/burning, 9.0%. PERFECT 2: erythema, 23.2%; scaling, 16.7%; dryness, 20.9%; stinging/burning, 12.9%) and severe 7.2% (PERFECT 1: erythema, 3.3%; scaling, 0.3%; dryness, 1.2%; stinging/burning, 3.0%. PERFECT 2: erythema, 7.2%; scaling, 3.0%; dryness, 2.5%; stinging/burning, 5.7%). The scores reached maximum severity at week 1 for the face and at Week 2-4 on the trunk; after these time points, scores diminished.

### Adverse Events

Most treatment emergent adverse events (TEAE5) related to trifarotene were cutaneous in nature and occurred at the application site. Severe AEs considered related to trifarotene therapy were reported in 6 subjects (PERFECT 1: 4 skin irritation, 1 sunburn, and 1 dermatitis allergic) and 3 subjects (PERFECT 2: 1 application site pain, 1 application site erosion, and 1 application site irritation) versus none in the vehicle group, but no AE was serious. AEs led to discontinuation in 1.9% (PERFECT 1) and 1.2% (PERFECT 2) of the trifarotene cream group and none in the vehicle group; these were most commonly application site irritation, allergic dermatitis, and skin irritation. There were no significant clinically relevant changes in vital signs, physical exams and laboratory parameters. There were 4/2420 (0.2%) pregnancies (one exposed to trifarotene cream and 3 to vehicle cream). The subject exposed to trifarotene was lost to follow-up.

### Discussion

Two large-scale phase III studies generated independent and substantial evidence of the efficacy and safety of trifarotene cream in moderate facial and truncal acne. In both anatomical regions (face and trunk), trifarotene cream was significantly superior to vehicle in success rates and in reduction of inflammatory and non-inflammatory lesion counts. There is sparse literature for the prevalence and treatment of chest and back acne despite it being a very common condition. Truncal acne often accompanies facial acne in adolescence, or it may first occur in and, indeed, persist well into, adulthood. Truncal acne has been estimated to occur in 56% of acne cases, with only a slightly higher predominance in males (55% vs 46%). Back acne, once thought to be a predominantly male disease, has shown to be prevalent in females.

The pathophysiologic mechanism of acne on the chest and back is similar to that of facial acne and centers on the physiology and properties of the pilosebaceous unit. Both anatomical areas are considered sebum-rich locations, although, the sebaceous follicles on the back have a different histologic appearance from those on the face. Few studies have evaluated drugs in the treatment of truncal acne and there are no well-designed comparative studies. Most studies have been small scale and not rigorously controlled. A review of the sparse evidence of the treatment outcomes of acne located in different anatomical regions has shown varying responses to systemic therapy when face and trunk are involved. The onset of effect of trifarotene 50 μg/g cream vs its vehicle was rapid, with significant reductions in both inflammatory and noninflammatory lesion counts seen as early as 1 week post treatment on the face, and as early as 2 weeks post treatment on the trunk. This observation is consistent with the findings of a 12-month, long-term safety study of trifarotene 50 μg/g cream, in which the success rate for the face and trunk demonstrated a consistent continuous clinical improvement over time and within the same subject (separate analysis).

Trifarotene had a manageable safety and tolerability profile, with the majority of adverse events being local cutaneous irritation mainly during the first weeks of treatment and improving thereafter. Local tolerability was better on the trunk than on the face. Similar results were reported in a long-term safety study of trifarotene.9 In the combined pool from both studies, the local signs and symptoms for the face and trunk that worsened post-baseline (skin dryness, erythema, scaling, stinging and burning) were mild to moderate in severity, with few subjects who experienced severe tolerability problems. The implementation of routine standard skin care such as non-comedogenic moisturizers, gentle cleanser and dosing regimen adjustments was sufficient to ensure treatment management and compliance in the majority of patients. There were no relevant changes in laboratory safety tests when trifarotene 50 μg/g cream was applied to large surface areas. Study limitations include absence of well-established optimal regimens and dosing strategies for truncal acne. These two phase 3 studies showed that once-daily trifarotene cream appears effective and safe, with manageable local tolerability, for the treatment for facial and truncal acne. The studies provide substantial evidence to support use of this new topical retinoid in facial and truncal acne.

Trifarotene 50 μg/g cream appears to be effective and safe in the treatment of moderate acne on the face and trunk, meeting all primary and secondary efficacy endpoints of two independent, randomized, well-controlled studies. Trifarotene exhibited the expected local tolerability profile of a topical retinoid. In both studies, the local tolerability profile of trifarotene was mostly mild to moderate and manageable when applied to the face as well as the larger body surface areas of the trunk.

### Example 2

### Determination of Long Term Safety and Efficacy of Trifarotene in the Treatment of Facial and Truncal Acne Patients

A multi-center, 52-week, double-blinded, and vehicle controlled group study (SATisFy) with once daily trifarotene 50 μg/g was conducted in subjects with moderate facial and truncal acne. The screening/baseline weeks were 1, 2, 4, 8, 12, 20, 26, 38 & week 52. Inclusion/exclusion criteria and assessment are as described in Example 1, unless mentioned otherwise. Additional assessments may include subject's facial acne assessment, quality of life (DLQI and CDLQI) and standard facial and trunk photography.

Primary Endpoint:

Safety
Local tolerability (erythema, scaling, dryness, stinging/burning) on Face & Trunk
Physical exam and Vital signs
Adverse events
Laboratory parameters

Secondary Endpoint:

Efficacy
Success rate: IGA/PGA score of 2 grade in improvement and Clear (0) or Almost Clear (1)
Grade change from baseline of IGA & PGA
Subject's assessment of facial acne improvement.

### Results

### Subject Disposition, Demographics, and Baseline Characteristics

Subject disposition data for SATisFy study are presented in Table 3. In SATisFy study, 507 subjects were screened and 453 received trifarotene treatment. Discontinuations were 107, and were primarily due to withdrawal by subject and lost to follow up

TABLE 3
Subject Disposition Data for SATisFy Study
All Subjects

CD5789 50 μg/g cream

| | n | (%) |
|---|---|---|
| Subject Screened | 507 | |
| Screen Failures | 52 | |
| Subjects Enrolled | 455 | |
| Subjects Treated | 453 | (99.6) |
| Completed the study: | 348 | (76.5) |
| 376 (83%) 6 months | | |
| 348 (76%) 360 days | | |
| Discontinued the study | 107 | (23.5) |
| Reason for discontinuation | | |
| Lack of efficacy | 4 | (0.9) |
| Adverse event | 16 | (3.5) |
| Subject's request | 53 | (11.6) |
| Protocol violation | 4 | (0.9) |
| Lost to follow-up | 17 | (3.7) |
| Other | 12 | (2.6) |
| Pregnancy | 1 | (0.2) |

Note:
Percentages are based on the number of subjects enrolled.

Demographic and baseline data for SATisFy study are presented in Table 4.

TABLE 4
Demographic and Baseline Data for SATisFy Study

| | |
|---|---|
| SAF population | SAFP population |
| (N = 453) | (N = 444) |

Age (years)

| | | | | |
|---|---|---|---|---|
| Mean (SD) | 18.3 | (6.6) | 18.4 | (6.5) |

| | | |
|---|---|---|
| Median | 16.0 | 16.0 |

| | | |
|---|---|---|
| (Min, Max) | (9.0, 54.0) | (9.0, 54.0) |

Age Group 1, n (%)

| | | | | |
|---|---|---|---|---|
| <18 years | 286 | (63.1) | 277 | (62.4) |
| 9 to 11 years | 18 | (4.0) | 9 | (2.0) |
| 12 to 17 years | 268 | (59.2) | 268 | (60.4) |
| ≥18 years | 167 | (36.9) | 167 | (37.6) |

Age Group 2, n (%)

| | | | | |
|---|---|---|---|---|
| Pediatric | 286 | (63.1) | 277 | (62.4) |
| 9 to 13 years | 65 | (14.3) | 56 | (12.6) |
| 14 to 17 years | 221 | (48.8) | 221 | (49.8) |
| Adult | 167 | (36.9) | 167 | (37.6) |
| 18 to 24 years | 109 | (24.1) | 109 | (24.5) |
| 25 to 64 years | 58 | (12.8) | 58 | (13.1) |

| | | |
|---|---|---|
| ≥65 years | 0 | 0 |

Gender, n (%)

| | | | | |
|---|---|---|---|---|
| Female | 226 | (49.9) | 217 | (48.9) |
| Male | 227 | (50.1) | 227 | (51.1) |

Race, n (%)

| | | | | |
|---|---|---|---|---|
| White | 432 | (95.4) | 424 | (95.5) |
| Black or African American | 12 | (2.6) | 11 | (2.5) |
| Asian | 3 | (0.7) | 3 | (0.7) |
| American Indian or Alaska | 1 | (0.2) | 1 | (0.2) |
| Native | | | | |
| Native Hawaiian or Other | 3 | (0.7) | 3 | (0.7) |
| Pacific Islander | | | | |
| Multiple | 2 | (0.4) | 2 | (0.5) |

Ethnicity, n (%)

| | | | | |
|---|---|---|---|---|
| Hispanic or Latino | 47 | (10.4) | 44 | (9.9) |
| Not Hispanic or Latino | 406 | (89.6) | 400 | (90.1) |

Skin Phototype, n (%)

| | | | | |
|---|---|---|---|---|
| Type I | 13 | (2.9) | 13 | (2.9) |
| Type II | 188 | (41.5) | 182 | (41.0) |
| Type III | 184 | (40.6) | 183 | (41.2) |
| Type IV | 53 | (11.7) | 52 | (11.7) |
| Type V | 7 | (1.5) | 7 | (1.6) |
| Type VI | 2 | (0.4) | 1 | (0.2) |
| Missing | 6 | (1.3) | 6 | (1.4) |

SAF: All Subjects who applied study drug at least once
SAFP: All Subjects who applied study drug once and that had PGA 3 acne at baseline

### Efficacy

Among patients treated with trifarotene, week 12 trunk IGA success rates were 26.6% and PGA success rates were 38.6%; and at week 52 success rates were 65.1% (IGA) and 66.9% (PGA) in trunk population as seen in FIG. 17. The overall success rate in same subject for truncal acne was 22.0% (87/396) at Wk12, 36.8%(138/375) at Wk20, 43.3%(157/363) at Wk26, 49.9% (177/355) at Wk38 and 57.9% (199/344) at Wk52.

Among patients treated with trifarotene for facial acne, the success rates were 41.4% (166/401) at Wk12, 54.8%(201/367) at Wk26 and 66.6%(233/350) at Wk52. 40.7%-45% (474/1214) subjects at Wk12 had a marked to complete improvement of Subject's Assessment of facial acne when treated with Trifarotene 50 μ/g cream compared with 25.3%-29.1% (303/1206) subjects in the vehicle cream group. Further, 53.8% (92/171) subjects at week 52 reported that acne had no effect on quality of life compared to 22.6% (47/208) at Baseline.

### Safety

### Local Tolerability

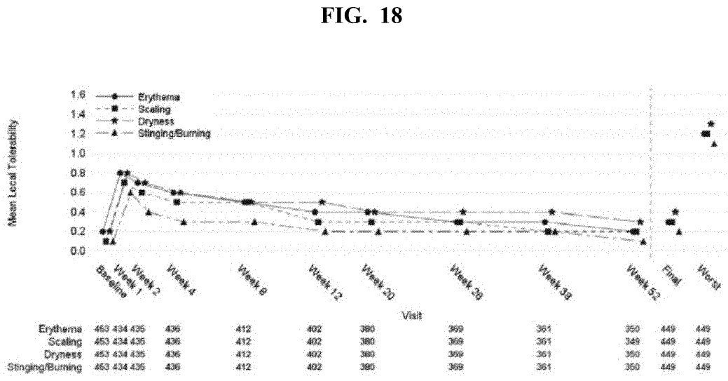

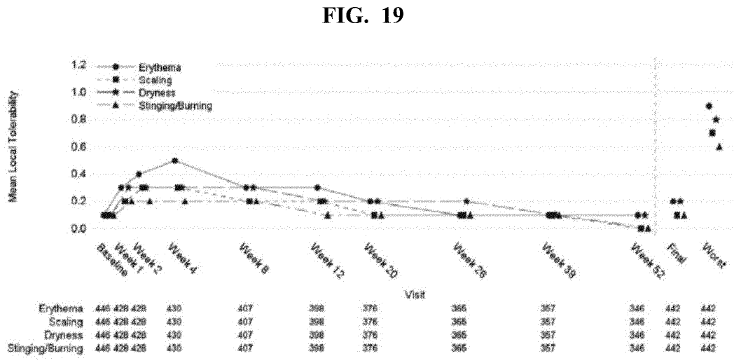

Local irritation related to trifarotene cream was transient and consistent with the known pattern of topical retinoid dermatitis (FIGS. 18-19; Severity scale score: 0 (none), 1 (mild), 2 (moderate), 3 (severe)). Local tolerability signs and symptoms related to trifarotene cream included erythema, scaling, dryness and stinging/burning. These were mostly mild to moderate by investigator assessment, with few being severe. The quarterly summary of treatment emergent adverse events (TEAS) in the safety study population is provided in Table 5. Summary of TEAS related to the study drug system organ class (SOC) and preferred term (PT), by quarter and overall is provided in Table 6.

TABLE 5
Quarterly Summary of TEAS in the Safety Study Population

| | CD5789 50 μg/g | | | | | | | | | |
|---|---|---|---|---|---|---|---|---|---|---|
| | (N = 453) | | | | | | | | | |
| | Q1 | | Q2 | | Q3 | | Q4 | | Overall | |
| | (M = 453) | | (M = 384) | | (M = 368) | | (M = 351) | | (N = 453) | |
| Number of TEAEs | 249 | | 91 | | 85 | | 43 | | 468 | |
| Subjects with any TEAE, | 154 | (34.0) | 68 | (17.7) | 62 | (16.8) | 36 | (10.3) | 218 | (48.1) |
| n (%) | | | | | | | | | | |
| Subjects with any TEAE | 46 | (10.2) | 8 | (2.1) | 9 | (2.4) | 2 | (0.6) | 57 | (12.6) |
| related to IP, n (%) | | | | | | | | | | |
| Subjects with any Cutaneous | 81 | (17.9) | 16 | (4.2) | 21 | (5.7) | 9 | (2.6) | 107 | (23.6) |
| TEAE, n (%) | | | | | | | | | | |
| Subjects with any Cutaneous | 46 | (10.2) | 8 | (2.1) | 9 | (2.4) | 2 | (0.6) | 57 | (12.6) |
| TEAE related to IP, n (%) | | | | | | | | | | |
| Subjects with any AESI, | 12 | (2.6) | 1 | (0.3) | 0 | | 0 | | 13 | (2.9) |
| n (%) | | | | | | | | | | |
| Subjects with any Serious | 4 | (0.9) | 0 | | 4 | (1.1) | 2 | (0.6) | 10 | (2.2) |
| TEAE, n (%) | | | | | | | | | | |
| Subjects with any Serious | 0 | | 0 | | 0 | | 0 | | 0 | |
| TEAE related to IP, n (%) | | | | | | | | | | |
| Subjects with any Severe | 5 | (1.1) | 1 | (0.3) | 2 | (0.5) | 1 | (0.3) | 9 | (2.0) |
| TEAE, n (%) | | | | | | | | | | |

| Subjects with any Severe | 2 | (0.4) | 1 | (0.3) | 0 | | 0 | 3 | (0.7) |
|---|---|---|---|---|---|---|---|---|---|
| TEAE related to IP, n (%) | | | | | | | | | |
| Subjects with any TEAE | 13 | (2.9) | 2 | (0.5) | 1 | (0.3) | 0 | 16 | (3.5) |
| leading to discontinuation, | | | | | | | | | |
| n (%) | | | | | | | | | |

| | | | | | | | | |
|---|---|---|---|---|---|---|---|---|
| Subjects with any TEAE | 12 | (2.6) | 1 | (0.3) | 0 | 0 | 13 | (2.9) |
| leading to discontinuation | | | | | | | | |
| related to IP, n (%) | | | | | | | | |

| | | | | | |
|---|---|---|---|---|---|
| Subjects with any TEAE | 0 | 0 | 0 | 0 | 0 |
| leading to death, n (%) | | | | | |
| Subjects with any TEAE | 0 | 0 | 0 | 0 | 0 |
| leading to death related | | | | | |
| to IP, n (%) | | | | | |

M = number of subjects at risk;
Q = quarter
Note:
Events are summarized by quarter based on study day of onset date: Q1 = 1 to 89, Q2 = 90 to 179, Q3 = 180 to 269, and Q4 = 270 to 391. A subject is considered at risk if their date of last study drug application is during or after the summarized period. Events with missing onset dates are not included in quarterly summaries, but are counted in the overall column.

TABLE 6
Summary of TEAS Related to the Study Drug System Organ Class
(SOC) And Preferred Term (PT), By Quarter and Overall

| | CD5789 50 μg/g | | | | | | | | | |
|---|---|---|---|---|---|---|---|---|---|---|
| | (N = 453) | | | | | | | | | |
| | Q1 | | Q2 | | Q3 | | Q4 | | Overall | |
| SOC, PT | (M = 453) | | (M = 384) | | (M = 368) | | (M = 351) | | (N = 453) | |
| Number of TEAEs related | 80 | | 10 | | 11 | | 2 | | 103 | |
| to the IP | | | | | | | | | | |
| Subjects with any TEAE | 46 | (10.2) | 8 | (2.1) | 9 | (2.4) | 2 | (0.6) | 57 | (12.6) |
| related to the IP, n (%) | | | | | | | | | | |
| General disorders and | 40 | (8.8) | 6 | (1.6) | 6 | (1.6) | 2 | (0.6) | 47 | (10.4) |
| administration site | | | | | | | | | | |
| conditions | | | | | | | | | | |
| Application site pruritus | 19 | (4.2) | 2 | (0.5) | 3 | (0.8) | 2 | (0.6) | 21 | (4.6) |

| Application site irritation | 17 | (3.8) | 1 | (0.3) | 1 | (0.3) | 0 | 19 | (4.2) |
|---|---|---|---|---|---|---|---|---|---|
| Application site dryness | 1 | (0.2) | 2 | (0.5) | 0 | | 0 | 3 | (0.7) |
| Application site erythema | 3 | (0.7) | 1 | (0.3) | 0 | | 0 | 3 | (0.7) |
| Application site pain | 2 | (0.4) | 0 | | 1 | (0.3) | 0 | 2 | (0.4) |
| Application site eczema | 0 | | 0 | | 1 | (0.3) | 0 | 1 | (0.2) |
| Injury, poisoning and | 6 | (1.3) | 1 | (0.3) | 2 | (0.5) | 0 | 9 | (2.0) |
| procedural complications | | | | | | | | | |
| Sunburn | 5 | (1.1) | 1 | (0.3) | 2 | (0.5) | 0 | 8 | (1.8) |

| Drug administered at | 1 | (0.2) | 0 | | 0 | 0 | 1 | (0.2) |
|---|---|---|---|---|---|---|---|---|
| inappropriate site | | | | | | | | |
| Skin and subcutaneous | 5 | (1.1) | 1 | (0.3) | 0 | 0 | 6 | (1.3) |
| tissue disorders | | | | | | | | |

| | | | | | | | |
|---|---|---|---|---|---|---|---|
| Acne | 3 | (0.7) | 0 | 0 | 0 | 3 | (0.7) |
| Rash | 1 | (0.2) | 0 | 0 | 0 | 1 | (0.2) |
| Skin burning sensation | 1 | (0.2) | 0 | 0 | 0 | 1 | (0.2) |

| | | | | | | | |
|---|---|---|---|---|---|---|---|
| Skin irritation | 0 | 1 | (0.3) | 0 | 0 | 1 | (0.2) |

| | | | | | | | |
|---|---|---|---|---|---|---|---|
| Blood and lymphatic system | 1 | (0.2) | 0 | 0 | 0 | 1 | (0.2) |
| disorders | | | | | | | |
| Lymphadenopathy | 1 | (0.2) | 0 | 0 | 0 | 1 | (0.2) |

| | | | | | | | |
|---|---|---|---|---|---|---|---|
| Infections and infestations | 0 | 0 | 1 | (0.3) | 0 | 1 | (0.2) |
| Skin Candida | 0 | 0 | 1 | (0.3) | 0 | 1 | (0.2) |

### Discussion

The SATisFy study generated independent and substantial evidence of the long term safety and efficacy of trifarotene cream in moderate facial and truncal acne. Most of the TEAEs occurred at the application site: signs and symptoms of worst peak for skin irritation occurred at wk1 (mild or moderate) for the face and trunk. Later decreasing & stabilizing. Trunk had a better local tolerability profile compared to the face as remained stable throughout the study duration. Well acceptable and manageable tolerability profile when trifarotene 50 μg/g cream was applied to large body surface areas of face and trunk for 12-week and 52-week period.

What is claimed is:

1. A method of treating acne vulgaris of the face and/or trunk in a subject in need thereof, the method comprising topically administering to the subject, a pharmaceutical composition comprising trifarotene or an equivalent thereof and a pharmaceutically acceptable carrier.

2. The method according to claim 1, comprising treating acne vulgaris of the trunk.

3. The method according to claim 1, wherein trifarotene or the equivalent thereof is administered once daily, twice daily, once per week, once every two weeks, once every three weeks, once every four weeks, once every five weeks, once every six weeks, once every seven weeks, or once every eight weeks.

4. The method according to claim 1, wherein the pharmaceutical composition is administered to the area of the subject affected with acne vulgaris.

5. The method according to claim 1, wherein the pharmaceutical composition is administered once daily to the affected areas of the subject.

6. The method according to claim 1, wherein the pharmaceutical composition comprises from about 1 μg/g to about 100 μg/g of trifarotene or an equivalent.

7. The method according to claim 1, wherein the pharmaceutical composition comprises about 50 μg/g of trifarotene or an equivalent thereof.

8. The method according to claim 1, wherein the pharmaceutical composition is formulated as a cream.

9. The method according to claim 1, wherein the pharmaceutical composition is formulated as an oil-in-water emulsion.

10. The method according to claim 1, wherein the amount of trifarotene or the equivalent thereof administered to the subject is about 0.00001 to about 1% by weight of the pharmaceutical composition.

11. The method according to claim 1, wherein the treatment comprises reduction in inflammatory lesion counts of the subject.

12. The method according to claim 1, wherein the treatment comprises reduction in noninflammatory lesion counts of the subject.

13. The method according to claim 1, wherein the acne vulgaris of the face and/or trunk comprises mild, moderate and severe acne vulgaris of the face and/or trunk.

14. The method according to claim 1, wherein the acne vulgaris of the face and/or trunk is moderate.

15. A pharmaceutical composition, comprising trifarotene or an equivalent thereof and a pharmaceutically acceptable carrier.

16. The pharmaceutical composition of claim 15, wherein the pharmaceutically acceptable carrier comprises one or more of an ointment, a cream, a gel, a spray, a foam, a paste or a suspension.

17. The pharmaceutical composition according to claim 1, comprising trifarotene or an equivalent thereof in an amount of from about 0.001 to about 0.1% by weight, relative to the weight of the total composition.

18. The pharmaceutical composition according to claim 1, comprising from about 1 μg/g to about 100 μg/g of trifarotene.

19. The pharmaceutical composition according to claim 1, formulated as a cream.
